(19) United States
(12) Patent Application Publication
(10) Pub. No.: US 2017/0182111 A1
(43) Pub. Date: Jun. 29, 2017

(54) Composition of extracts from the genus Hippeastrum and the use of their components for the production of pharmaceuticals and dietary supplements

(71) Applicant: BERBEE BEHEER BV., LR Lisse (NL)

(72) Inventors: Strahil Hristov Berkov, Ihtiman (BG); Jaime Bastida Armengol, Gava (ES); Carlos Codina Mahrer, Barcelona (ES); Jean Paulo De Andrade, Caxias do Sul, Rio Grande do Sul (BR); Rudolf Leonardus Maria Berbee, RZ Voorhout (NL)

(21) Appl. No.: 14/910,116

(22) PCT Filed: Oct. 9, 2013
(86) PCT No.: PCT/BG2013/000046

§ 371 Date: Feb. 4, 2016

(30) Foreign Application Priority Data
Mar. 7, 2013 (BG) 111420

(51) Int. Cl. A61K 36/88; B01D 15/36; B01D 11/04; A61K 31/407; A61K 31/55

(52) U.S. Cl. CPC A61K 36/88; A61K 31/407; A61K 31/55; B01D 11/04; B01D 15/361; A61K 2121/00

(57) ABSTRACT

A composition of extracts from the genus Hippeastrum and their components is intended for the production of pharmaceuticals and dietary supplements. The composition consists of galanthamine from 40% to 98%, haemanthamine from 0.01% to 35%, narwedine from 0.01% to 10%, and/or other alkaloids of homolycorine-, and/or tazettine-, and/or monthanine- and/or haemanthamine- and/or galanthamine- and/or lycorine-type from 0.001% to 35%, and non-alkaloid plant metabolites from 0.001 to 1%, derived from the total extract of the genus Hippeastrum. The alkaloid content can vary from 0.01% to 99% and non-alkaloid plant metabolites and/or other inactive excipients and/or solvents from 1% to 99.9%, respectively. The composition may be in dry or liquid form. It is used to isolate pure compounds of haemanthamine and galanthamine.

### CROSS-REFERENCE TO RELATED APPLICATIONS

This application is a Section 371 National Stage Application of International Application No. PCT/BG2013/000046, filed Oct. 9, 2013, which is incorporated by reference in its entirety and published as WO 2014/134692 on Sep. 12, 2014, in English.

### BACKGROUND

The invention concerns the composition of extracts from the genus Hippeastrum and the use of their components for the production of pharmaceuticals and dietary supplements in pharmaceutical and food industries.

It is known from the state of the art that the species of the genus Hippeastrum (Amaryllidaceae) are large tropical plants that show significant variation in their alkaloid composition (Rev. Latinoamer. Qufm. 2012, 40: 83). It is also known that there is a significant intraspecific variation in the composition and concentration of alkaloids in different species of the family Amaryllidaceae such as Leucojum aestivum, Galanthus elwesii, and G. nivalis (Chem. Biodiv. 2011, 8: 115; Biochem. Syst. Ecol., 2013, 46: 152), but no evidence of such a variation within species or cultivars of the genus Hippeastrum has been found.

From the state of the art the composition of an extract from the genus Hippeastrum is known to include the alkaloids: galanthamine, galanthine, lycorine, hippamine, narcissidine, hippeastrine, haemanthamine, crinidine, powelline and tazettine (Archiv der Pharmazie and Berichte der Deutschen Pharmazeutischen Gesellschaft 1962, 295: 920).

From the state of the art, the composition of an extract from the genus Hippeastrum is known to include the alkaloids: 2, 7-dimethoxyhomolycorine, candimine, tazettine, pretazettine, epimacronine, haemanthamine, hamayne and triphaeridine (Magn. Res. Chem. 2011, 49: 668).

The composition of an extract from the genus Hippeastrum is also known to include the alkaloids: hippadine, hippacine, hippeastrine, vittatine, pancracine and tazettine (Pianta Medica 1975, 28: 336).

To the best of our knowledge, there are no data on the percentage ratio of the alkaloids in the alkaloid composition of extracts from the genus Hippeastrum measured by an analytical technique such as gas chromatography-mass spectrometry (GC-MS) or high-pressure liquid chromatography (HPLC).

There are no data in the literature on the industrial production of galanthamine from the genus Hippeastrum.

Galanthamine (chemical formula A) is a known, natural alkaloid produced by plants of the family Amaryllidaceae, including the relatively small plants of the genera Galanthus, Narcissus, Leucojum and Lycoris (Biotechnol. & Biotechnol. Eq. 2009: 1170). Galanthamine is a reversible cholinesterase inhibitor (AChE) that has analgesic and antioxidant properties. It is used to treat Alzheimer's disease (Drugs 2000, 60: 1 095) and dependence on alcohol, drugs and nicotine (U.S. Pat. No. 5,643,905). WO/201 0/025485 patent describes the use of galanthamine hydrobromide to produce drugs to treat glaucoma. EP0236684 patent describes the use of galanthamine or its analogues for the treatment of Alzheimer's disease. According to patent WO/2006/099635, galanthamine is isolated from biological material, preferably from plants of the genus Galanthus, Narcissus, Leucojum and/or Lycoris.

It is also known that dietary supplements containing galanthamine, and in particular Leucojum aestivum extract, are used to improve muscle strength and endurance in athletes (WO2000041540).

Narwedine (chemical torula B) is another known reversible cholinesterase inhibitor (Proteins 2001, 42: 182), used as a precursor for the synthesis of galanthamine (WO2005/030713).

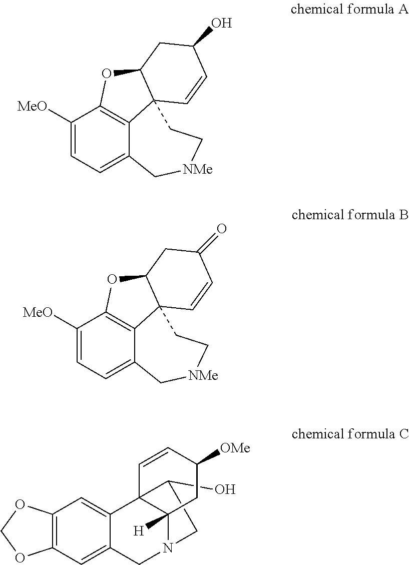

Haemanthamine (chemical formula C) is another known alkaloid. This lead molecule has been shown to be a potent inducer of apoptosis in tumor cells at micromolar concentrations (Phytochemistry 2007, 68: 1 068), and consequently has been the subject of intense research in recent years.

The disadvantages of the known compositions obtained from plant sources of galanthamine, described in patent WO/2006/099635, are that they contain significant amounts of accompanying alkaloids that influence its isolation, and/or have toxic effects, so the direct use of extracts from these plants for the preparation of pharmaceuticals and dietary supplements is undesirable. For example, in different populations of Leucojum aestivum, galanthamine ranges from 0.03 to 0.4% of the dry weight, and is accompanied by substantial amounts of lycorine, homolycorine and 8-O-demethylhomolycorine (Biochem. Syst. Ecol., 2013, 46: 152). The Narcissus pseudonarcissus cultivar “Carlton”, grown for the production of galanthamine, is mainly propagated vegetatively and therefore has low genetic variability (Hanks G. 2002, Narcissus and daffodil, the genus Narcissus, Taylor & Francis, London and New York, p. 53). Only the bulbs from this Narcissus cultivar are used as raw material for galanthamine production but they also contain significant quantities of haemanthamine and narciclasine (Phytochemistry 2013, 88: 43). Galanthamine, lycorine and lycorenine, along with eleven other alkaloids, were isolated from Lycoris radiata bulbs (Helv. Chim. Acta 2011, 94: 178), a plant cultivated in China as a galanthamine source.

The main disadvantages of the plants described in patent WO/2006/099635 as sources of galanthamine are their small size, short growing period and long period of physiological dormancy.

### SUMMARY

The disclosure provides the composition of extracts from the genus Hippeastrum, which contain the following components as determined by GC-MS: galanthamine from 40% to 98%, haemanthamine from 0.01% to 35%, narwedine from 0.01% to 10%, and/or other alkaloids from homolycorine, and/or tazettine, and/or montamine, and/or narciclasine and/or lycorine type from 0.001% to 35% and/or nonalkaloid plant metabolites from 0.001% to 1%, derived from total extracts of the genus Hippeastrum.

The composition of an extract from the genus Hippeastrum, the subject of this disclosure, may be part of a dry or liquid totally or partially purified extract, and obtained by a suitable extraction method and solvent. The non-alkaloid components of the totally or partially purified extracts from the genus Hippeastrum can range from 1 to 99%.

### BRIEF DESCRIPTION OF THE DRAWINGS

FIG. 1, GC-MS chromatogram of the composition of an extract from unselected plants of Hippeastrum, in which the three main alkaloids of the composition are indicated;

FIG. 2, GC-MS chromatogram of composition of an extract from unselected plants of the genus Hippeastrum, in which the three main alkaloids of composition are indicated;

FIG. 3, GC-MS chromatogram of the composition of an extract from unselected plants of the genus Hippeastrum;

FIG. 4, GC-MS chromatogram of the composition of an extract from selected plants of the genus Hippeastrum, in which the three main alkaloids are indicated;

FIG. 5, GC-MS metabolic profile of a total methanol extract from the genus Hippeastrum.

### DETAILED DESCRIPTION

The composition of extracts from the genus Hippeastrum is obtained from selected whole plants or plant organs grown outdoors or in greenhouses in different geographical regions by conventional technologies, or as hydroponic cultures or by biotechnological methods. Feedstock is washed with clean water and allowed to dry in the air. After that the plants are cut mechanically to a size of 1-5 cm and subjected to drying in traditional equipment for drying fruits with circulating warm air. Drying is at a temperature of 15° to 85° C. for 5 to 96 hours until the final moisture content of the biomass is less than 15%. When working with fresh plant material, it can be directly subjected to extraction after cutting. The plant material is ground to a particle size from 85 microns to 3 mm using a conventional industrial plant mill.

To obtain the composition of the extracts from the genus Hippeastrum and the components thereof, the plant material can be subjected to conventional procedures for liquid-liquid extraction of alkaloids. Dried and finely ground whole plants or plant organs (leaves, bulbs and roots) or fresh plants containing between 0.05% and 2.0% of galanthamine in the dry herbage are loaded into a system of percolators and extracted with 0.1-5% (v) aqueous solution of an inorganic acid (eg. sulfuric, phosphoric, etc.) with countercurrent. The primary extract is made alkaline with 1-10% aqueous solution of potassium hydroxide or other bases to pH 8-9 and extracted with water-insoluble organic solvents such as chloroform, dichloromethane, etc. The organic solvent is washed with water and then alkaloids are extracted with 0.1-5% (v) aqueous solution of an inorganic acid. After basification of the aqueous solution with 1-10% aqueous solution of potassium hydroxide or other bases to pH 8-9, the composition of the extract from the genus Hippeastrum and its components, the subject of the disclosure, is obtained by extraction with a water insoluble organic solvent such as chloroform, dichloromethane, hexane, benzene, ethyl acetate, etc. After evaporation of the solvent under vacuum, the alkaloids present in the of the extracts from the genus Hippeastrum are in the form of bases. They can be converted into pharmaceutically acceptable salts by the addition of a suitable acid before evaporation of the organic solvent. Other chemical or physical modification of the composition is allowed, such as derivatization (eg. esterification) or freezing to obtain by-products with altered physical and/or chemical properties.

The composition of the extracts from the genus Hippeastrum and the components thereof, the subject of this disclosure, may be obtained by other conventional methods such as maceration, or obtained by microwave, ultrasonic, or super-critical carbon dioxide extraction and/or membrane technologies for extraction. Various organic solvents and water can be used for extraction, controlling the pH from 1 to 12 with various organic and inorganic acids and bases.

Isolation and purification of the individual components of the extracts from the genus Hippeastrum can be achieved by chromatographic methods (such as preparative liquid chromatography, partition chromatography, etc.).

The galanthamine and alkaloid composition mentioned above can be used in free form or in salt form, preferably in pharmaceutically acceptable salt form, for example, obtained by adding inorganic or organic acids, for example, hydrobromic acid. It can be also used after chemical or physical modifications.

Quality and quantity control of the plant material and the composition of the extracts from the genus Hippeastrum and the components thereof is performed by a method described in the literature (Talanta 2011, 83: 1455). Dried plant material from the genus Hippeastrum (50 mg) is extracted with 1 ml of methanol containing 50 micrograms of codeine as an internal standard. The mixture is extracted at 60° C. for 30 minutes and after centrifugation, 800 microliters of the supernatant are separated and evaporated. The resulting solids are dissolved in 500 microliters of 2% H2S04 and extracted three times with 500 microliters of chloroform. Then the aqueous phase is made alkaline to pH 8-9 with 25% solution of ammonia and the alkaloids are extracted with chloroform. After evaporation of the organic solvent, the dry extract is dissolved in methanol prior to gas chromatography-mass spectrometry.

The composition of the extracts from the genus Hippeastrum and the components thereof are used in the production of drugs for the prevention or treatment of various diseases controlled with AChE inhibitors. These include mild to moderate Alzheimer's disease, myopathy, sensory and motor dysfunction, conditions associated with disorders of the central nervous system and others. For the above mentioned diseases and conditions, the required dose varies depending on the method of application and desired effect. Drugs are administered orally, for example, in the form of water-ethanol tinctures, tablets, coated tablets, capsules, oral sprays or sub-lingual agents, intranasal, in the form of nasal sprays, transdermal, or topical, for example, in the form of lotions, creams, ointments, skin patches or injectables. Preferably, the pharmaceuticals are in the form of a single dose. Each dose comprising the composition of extracts from the genus Hippeastrum or the components thereof may contain from 1.0 mg to 20 mg galanthamine.

The composition of the extracts from the genus Hippeastrum and the components thereof, the subject of this disclosure, are used in the manufacture of dietary supplements, which can be in the form of tablets, capsules or solutions. The composition of the extracts from the genus Hippeastrum and the components thereof are used in the production of dietary supplements for improving the quality of life of healthy people or to improve muscle strength and endurance.

The composition of the extracts from the genus Hippeastrum and the components thereof are also used in the production of pure substances of galanthamine, narwedine and haemanthamine, which are applied in the pharmaceutical industry and clinical research.

Pharmaceuticals and dietary supplements may contain from 0.01% to 99.9% of the composition of the extracts from the genus Hippeastrum and the components thereof. The other ingredients of the pharmaceuticals and dietary supplements can include plant metabolites (sugars, lipids, amino acids, organic acids, etc.), excipients such as lactose monohydrate, maltodextrin, water, ethanol or mixtures thereof.

The advantage of the composition of the extracts from the genus Hippeastrum and the various components thereof is that they are derived from selected, large and evergreen species and cultivars of the genus Hippeastrum. The extracts contain a high percentage of galanthamine and small amounts of nawedine, haemanthamine and other minor alkaloids, which facilitates the purification of galanthamine. The other alkaloids have low toxicity or no toxicity, allowing the composition of the extracts from the genus Hippeastrum to be used for the production of pharmaceuticals and dietary supplements. The composition of the extracts from the genus Hippeastrum and the components thereof can be used as a source for the industrial production of galanthamine and haemanthamine.

The invention is illustrated by the following examples of performance without limiting it

### Example 1

The disclosure provides the composition of an extract from the genus Hippeastrum containing the following components measured by GC-MS: galanthamine 54.1%, narwedine 1.7%, haemanthamine 19.5%, an alkaloid of homolycorine type 13.6%, other alkaloids 10.7% and non-alkaloid metabolites 0.4% (Table 1).

Five grams of the raw material—powdered dry herbage from unselected plants of the species Hippeastrum papillio, were extracted three times for 24 hours with 40 ml of 2% aqueous solution of sulfuric acid (H2S04). The aqueous solutions were combined and made alkaline with ammonia to pH 8-9, and then the resulting aqueous fraction was extracted twice with 30 ml of chloroform. The combined chloroform fraction was washed with 20 ml of water and then the organic solvent was extracted three times with 20 ml of 2% aqueous solution of H2S04. The alkaloids were obtained from the aqueous phase after bazification with ammonia to pH 8-9 and two-time extraction with 20 ml of chloroform. After evaporation of the organic solvent the dry composition of extract from the genus Hippeastrum was dissolved in methanol before GC-MS analysis.

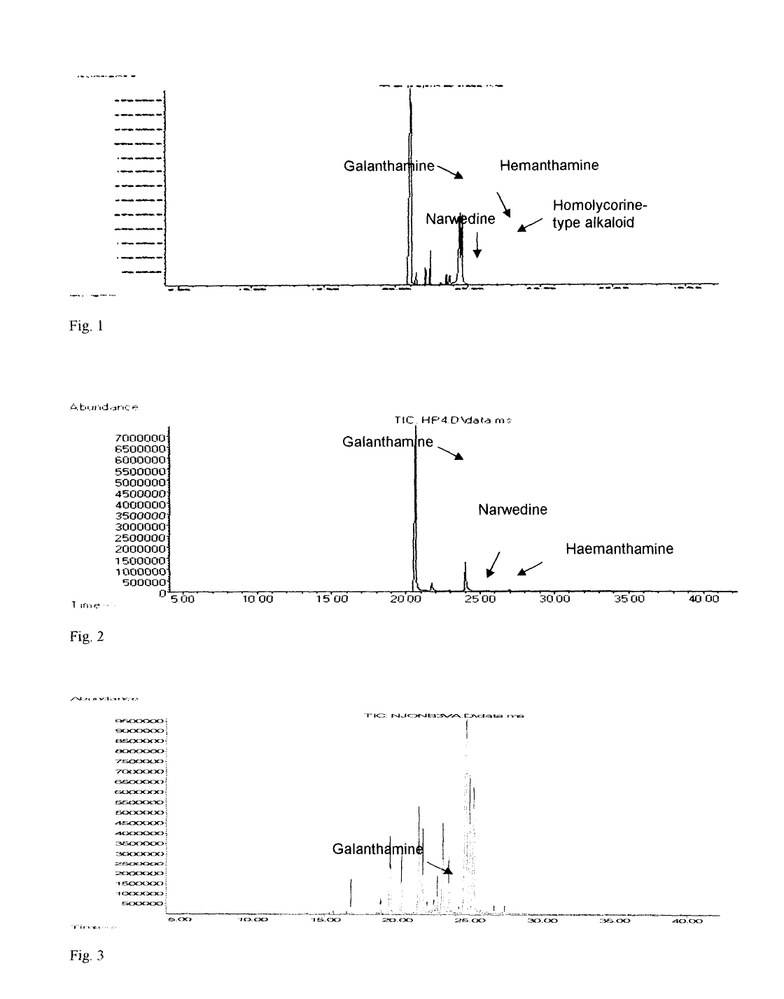

Quality and quantity control of the plant material and the composition of extract from the genus Hippeastrum is performed by a method described above by GC-MS. Data from the analysis are presented in FIG. 1 and Table 1.

TABLE 1
Examples of the composition of extracts from the genus Hippeasrtum and the components
thereof derived from plants of different origins. The content of the main alkaloids in the
composition is expressed as a percentage based on GC-MS analyses.

| | | | | | |
|---|---|---|---|---|---|
| | | | Hemanthamine | Other | |
| Plant (code) | Galanthamine % | Narwedine % | % | alkaloids % | Non-alkaloids |

Unselected plants

| | | | | | | |
|---|---|---|---|---|---|---|
| 1 | H. papilio (PAP) | 54.1 | 1.7 | 19.5 | 24.3 | 0.4 |
| 2 | H. papilio (HPB) | 62.5 | 5.9 | 22.5 | 8.0 | 0.1 |
| 3 | H. papilio (HBR) | 88.4 | 1.3 | 9.6 | 0.7 | Not detected |
| 4 | H. papilio (HRE) | 83.2 | 3.8 | 8.3 | 4.6 | 0.1 |
| 5 | H. vittatum (HIV) | 4.6 | 0.1 | 0.9 | 93.6 | 0.8 |

Selected plants

| | | | | | | |
|---|---|---|---|---|---|---|
| 5 | H. papilio (HP1) | 94.3 | 1.5 | 3.7 | 0.4 | 0.1 |
| 6 | H. papilio (HP2) | 90.1 | 1.7 | 4.1 | 3.4 | 0.7 |
| 7 | H. papilio (HP13) | 95.6 | 1.2 | 1.9 | 1.3 | Not detected |
| 8 | H. papilio (HP14) | 97.8 | 0.7 | 0.8 | 0.3 | 0.4 |
| 9 | H. papilio (HP17) | 91.4 | 1.7 | 6.4 | 0.3 | 0.2 |

### Example 2

Prepared as in Example 1, but with unselected plants of Hippeastrum papillio from a different origin than in Example 1. The invention provides a composition of an extract from the genus Hippeastrum and its components, which include the following: galanthamine 88.4%, narwedine 1.3%, haemanthamine 9.6%, and other alkaloids 0.7%. Data from the analysis are presented in FIG. 2 and Table 1.

### Example 3

Prepared as in Examples 1 and 2, but with unselected plants from Hippeastrum vittatum, whose extract contains the following components: galanthamine 4.6%, narwedin 0.1%, haemanthamine 0.9%, other alkaloids 93.6% and non-alkaloid compounds 0.8%. The results are presented in FIG. 3 and Table 1.

Table 1 presents other results from the analysis of the composition of extracts from unselected plants of the genus Hippeastrum, like Examples 1-3. These results show qualitative and quantitative variation in the alkaloid profiles, respectively, in the composition of extracts from the genus Hippeastrum obtained from unselected plants and plants of different origin. This variability allows appropriate selection of genotypes from different species and of different origin within the species with desirable properties and extract composition.

### Example 4

Prepared as in Examples 1-3, but with selected plants of Hippeastrum papillio whose extract contains the following components: galanthamine 97.8%, narwedine 0.7%, haemanthamine 0.8%, other alkaloids 0.3%, and non-alkaloid compounds 0.4%.

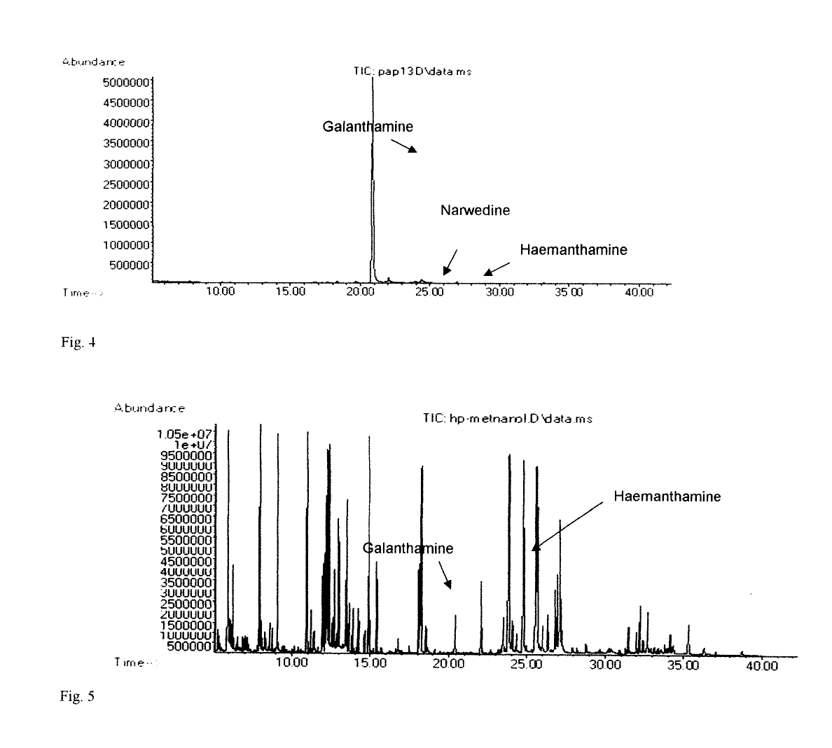

The results are presented in FIG. 4 and Table 1.

Table 1 presents the results of other analyses of selected plants, as in Example 3. These results show the potential of selected plants to provide an extract composition from the genus Hippeastrum, whose components have certain characteristics, namely high levels of galanthamine.

### Example 5

The disclosure provides a total alcoholic extract, which is used to obtain the composition of an extract from the genus Hippeastrum and its components.

Five grams of powdered plant material is macerated in 35 ml of methanol for 48 hours at room temperature. After filtration under pressure, the plant material is washed with two portions of 15 ml of methanol, which are combined with the filtrate in a total methanol extract.

Aliquots of 50 microliters of the resulting total extract is transferred to gas chromatography vials and evaporated in a vacuum evaporator. The dry extract is dissolved in pyridine (50 microliter) and derivatized for 90 min at 60° C. with 50 microliters of BSTFA (N,O-bis(trimethylsilyl)trifluoroacetamide). The derivatized extracts are diluted with 300 microliters of chloroform before analysis with GC-MS.

The rest of the total methanol extract is concentrated under vacuum to give a total dry extract. This extract is dissolved in 30 mf of 2% aqueous solution of H2S04 and extracted three times with 15 ml of chloroform. Then, the aqueous solution is made alkaline with ammonia to pH 8-9 and the alkaloids are extracted twice with 20 ml of chloroform. The combined chloroform extracts are evaporated under vacuum to obtain a dry alkaloid fraction representing the composition of the extract from the genus Hippeastrum. The alkaloid profiles from GC-MS analyses are similar to those from Examples 1 to 4, depending on the origin of the sample.

Metabolic profile of the resulting total methanol extract is shown in FIG. 5.

1-8. (canceled)

9. An extract derived by liquid-liquid or ion-exchange extraction of the total dry or liquid extracts wherein the total dry or liquid extracts are from selected Hippeastrum papilio plants and include alkaloids comprising galanthamine from 70% to 98%, and haemanthamine from 0.01% to 20%, narwedine from 0.01% to 8% wherein the percentages are expressed as a percent of the integrated GC-S peak areas.

10. The extract of claim 9 and wherein the alkaloids further comprise alkaloids of homolycorine-, and/or tazettine-, and/or monthanine- and/or lycorine-type from 0.001% to 28%, and/or non-alkaloid plant metabolites from 0.001% to 1%.

11. The extract of claim 9 and wherein the total dry or liquid extracts from selected Hippeastrum papilio plants are obtained by aqueous, alcoholic or super-critical carbon dioxide extraction and are characterized by an alkaloid content that may vary from 0.01% to 99% (by weight) and by non-alkaloid plant metabolites and/or other inactive excipients and/or solvents from 1% to 99% (by weight).

12. The extract of claim 9 and wherein the alkaloids are in the form of pharmaceutically acceptable salts or free bases, or may include chemical modifications comprising esterification and oxidation.

13. The extract of claim 9 and further comprising pure compounds of haemanthamine and galanthamine isolated from the Hippeastrum papilio extract.

14. The extract of claim 9, and further comprising adding the extract to pharmaceuticals and dietary supplements wherein the pharmaceuticals and dietary supplements can be used for the prevention and treatment of diseases and conditions that respond to prevention and treatment with acetylcholinesterase inhibitors, including mild to moderate vascular dementia, Alzheimer's disease, myopathy, muscle performance, sensory and motor dysfunction of the central nervous systems.

15. The extract of claim 9 wherein the extract is derived from plants of Hippeastrum papilio (Ravenna) Van Scheepen (Amaryllidaceae) including its hybrids, cultivars, offsprings and tissue cultures with different ploidy levels.

16. A method of forming an extract, the method comprising
providing a selected amount of Hippeastrum papilio plant material; and
subjecting the selected amount of Hippeastrum papilio plant material to a liquid-liquid or ion-exchange extraction to obtain an extract that include alkaloids comprising galanthamine from 70% to 98%, and haemanthamine from 0.01% to 20%, narwedine from 0.01% to 8% wherein the percentages are expressed as a percent of the integrated GC-S peak areas.

17. The method of claim 16 and wherein the alkaloids in the extract further comprises alkaloids of homolycorine-, and/or tazettine-, and/or monthanine- and/or lycorine-type from 0.001% to 28%, and/or non-alkaloid plant metabolites from 0.001% to 1%.

18. The method of claim 16 and wherein subjecting the selected amount of Hippeastrum papilio plant material to a liquid-liquid or ion-exchange extraction comprises aqueous, alcoholic or super-critical carbon dioxide extraction and are characterized by an alkaloid content that may vary from 0.01% to 99% (by weight) and by non-alkaloid plant metabolites and/or other inactive excipients and/or solvents from 1% to 99% (by weight).

19. The method of claim 16 and further comprising esterifying or oxidizing the alkaloids to form of pharmaceutically acceptable salts or free bases.

20. The method of claim 16 and further comprising refining the extract to substantially pure compounds of haemanthamine and galanthamine from the Hippeastrum papilio extract.

21. The method of claim 16, and further comprising adding the extract to pharmaceuticals and dietary supplements wherein the pharmaceuticals and dietary supplements can be used for the prevention and treatment of diseases and conditions that respond to prevention and treatment with acetylcholinesterase inhibitors, including mild to moderate vascular dementia, Alzheimer's disease, myopathy, muscle performance, sensory and motor dysfunction of the central nervous systems.

22. The method of claim 16 and wherein providing a selected amount of Hippeastrum papilio plant material comprises providing plants of Hippeastrum papilio (Ravenna) Van Scheepen (Amaryllidaceae) including its hybrids, cultivars, offsprings and tissue cultures with different ploidy levels.
